(19) United States
(12) Patent Application Publication
(10) Pub. No.: US 2008/0020466 A1
(43) Pub. Date: Jan. 24, 2008

(54) Detection Method Using Detecting Device

(75) Inventors: Sachio Nomura, Kawagoe-shi (JP); Masatoshi Kondo, Kawagoe-shi (JP); Kazumi Kenmotsu, Kawagoe-shi (JP); Makoto Nagano, Kawagoe-shi (JP); Yukiko Sagehashi, Kawagoe-shi (JP); Yoko Kimura, Kawagoe-shi (JP); Toru Egashira, Kawagoe-shi (JP)

Correspondence Address: SNIDER & ASSOCIATES, P. O. BOX 27613, WASHINGTON, DC, 20038-7613, US

(73) Assignee: BML, INC., Tokyo (JP)

(21) Appl. No.: 11/660,372

(22) PCT Filed: Aug. 17, 2004
(86) PCT No.: PCT/JP04/12043

§ 371 Date: Aug. 16, 2007

(51) Int. Cl. G01N 33/00; B01L 11/00

(52) U.S. Cl. 436/2; 422/61

(57) ABSTRACT

Provided is a detection method employing a detection instrument having a plurality of wells at a surface thereof, the method including depositing a detection object, preferably in coexistence with a specific holding substance, onto the inner walls of the wells of the detection instrument; bringing a reaction solution into contact with the inner walls of the wells of the detection instrument on which the detection object has been deposited, the reaction solution containing a substance reactive with the detection object; and detecting a signal generated through this contact, to thereby evaluate the detection object. In this method, a liquid phase reaction can be efficiently performed in the detection instrument for carrying out a biological reaction (e.g., a microarray) so that samples contained in the wells are not mixed with one another, and that air bubbles are prevented from being generated in the wells.

### TECHNICAL FIELD

The present invention relates to a biological detection method employing, in particular, a detection instrument having a microstructure.

### BACKGROUND ART

In the fields of medicine and biology, decoding the primary structure of the human genome is one of the most remarkable achievements of the 20th century.

Results of such genome analysis are envisaged to be employed in a variety of industrial fields and to contribute to dramatic technological developments.

The human genome has been found to contain a variety of polymorphic markers, and the vast majority thereof are single nucleotide polymorphisms (SNPS). SNPs are said to account for 80% or more of all polymorphic markers. At present, hopes have been heightened for applications of SNPs in research of disease-related genes and subsequent development of new drugs; i.e., development of drugs based on the human genome.

In addition, detection of SNPs is expected to be useful for, for example, analyzing an individual's physical constitution, and thus will pave the way for so-called personalized medicine.

Currently, in order to improve efficiency of gene analysis, microarray plates have been provided. By use of such microarray plates, hybridization or a similar reaction with a very large number of nucleotide fragments is performed on a very small chip, leading to elucidation of the function, etc. of specific genes. Thus, through use of such microarray plates, hybridization reactions which must be performed for many different combinations of fragments can be performed efficiently, and the amount of a sample employed can be considerably reduced as compared with the case where a conventional technique is employed.

A currently employed microarray technique in which a uniform solution prepared from a sample is applied in one time onto a chip including a microarray plate on which a variety of probes and targets have been immobilized, to thereby perform hybridization is efficient analysis means which enables different sequences in the uniform solution to be analyzed in a single step.

Employment of a microarray plate having, on its surface, very small wells provided at high density (each of the wells enables liquid phase reaction to be performed therein) requires an apparatus capable of dispensing an object on the order of nanoliter (nL) with an accuracy on the order of micrometer (μm) (hereinafter such an apparatus may be referred to as a “microdispenser”) (although dispensing a reaction reagent into only a few wells can be performed through a manual method, much difficulty is encountered in treating numerous wells with the manual method). Examples of microdispensers which may be employed include inkjet-type microdispensers such as synQUAD™ (product of Cartesian). Specifically, an inkjet-type microdispenser is required for dispensing different reagents into individual reaction wells. Conventionally, a microdispenser has been required for dispensing a single reagent into wells provided on the entire surface or a large area of a plate.

As has been known, in a detection process employing a microarray plate requiring such a microdispenser, a very small matter could cause measurement error. For example, when different types of samples, etc. are dispensed onto a microarray plate, in order to prevent mixing of different sample solutions in a nozzle or piping of the microdispenser, a washing process must be performed before suction of the respective sample solutions, which requires a period of time. This time-requiring process raises a problem in terms of a great difference in volume, due to evaporation, between the initially dispensed sample and the finally dispensed sample.

In a method for solving such a problem, firstly, a sample, etc. are dispensed into microwells of a plate (first dispensing), followed by drying, and subsequently, a single reaction solution is collectively and rapidly dispensed into the wells containing the dried sample (second dispensing). This method can considerably reduce the possibility of a difference in volume between the solutions contained in the wells. Through the second dispensing, the sample, etc. which have been dried after the first dispensing are eluted and brought into contact with the solution, whereby a target detection reaction is initiated.

In this method, in order to prevent drying after the second dispensing, for example, a sealing material for prevention of evaporation is provided so as to cover the microwells. In this case, the sealing material must be brought into close contact with the surface of the plate so as to prevent mixing (due to contact) of the solutions contained in the microwells.

However, during the course of sealing, air bubbles could enter between the sealing material and a liquid reaction system contained in each of the microwells, leading to occurrence of detection error. When, for example, the reaction solution is dispensed into the microwells so that each of the microwells is filled with the solution and no air bubbles enter the microwell, there is a high likelihood that the solutions contained in adjacent microwells overflow the microwells, and then come into contact and mix with one another. Even in such a case, there is also a high likelihood that air bubbles enter the microwells.

In view of the foregoing, an object of the present invention is to provide means for efficiently performing a liquid phase reaction in a detection instrument for carrying out a biological reaction (e.g., a microarray) so that samples contained in microwells are not mixed with one another after the aforementioned first dispensing, and that air bubbles are prevented from being generated in the microwells.

### DISCLOSURE OF THE INVENTION

In order to solve the aforementioned problems, the present inventors have conducted extensive studies, and as a result have found that the problems can be solved by depositing, through first dispensing, an object to be detected (hereinafter may be referred to as a “detection object”) (e.g., a sample) onto the inner walls of a plurality of microwells provided on a detection instrument (e.g., a microarray plate), and, within a short period of time, bringing a reaction solution into contact with the detection object.

Accordingly, the present invention provides a detection method employing a detection instrument having a plurality of wells at a surface thereof, the method comprising depositing a detection object onto the inner walls of the wells of the detection instrument; bringing a reaction solution into contact with the inner walls of the wells of the detection instrument on which the detection object has been deposited, the reaction solution containing a substance reactive with the detection object; and detecting a signal generated through this contact, to thereby evaluate the detection object (hereinafter the method may be referred to as “the present detection method”).

In a preferred mode of the present detection method, the detection object is caused to coexist with a specific substance on the inner walls of the microwells. In this mode, an allowable operation period of time from the second dispensing to detection can be prolonged by providing a predetermined interval between the time at which the reaction solution comes into contact with the inner walls and the time at which the reaction solution comes into direct contact with the detection object deposited on the inner walls, to thereby facilitate detection of a target biological reaction.

In a specific mode of the present detection method provided by the present invention, the detection object deposited on the inner walls of a plurality of the wells provided at the surface of the detection instrument is caused to coexist with (1) a substance which, when being itself or in the form of a mixture with a solvent, undergoes change in phase from solid to liquid at about 10 to about 90° C. (hereinafter the substance may be referred to as a “temperature-responsive substance”), and/or (2) a substance which is gradually dissolved in a solvent at room temperature (hereinafter the substance may be referred to as a “gradually soluble substance”); and the substance (1) and/or the substance (2) is dissolved in the reaction solution during detection.

In the present detection method, preferably, a sealing material is provided so as to cover the wells after the second dispensing. (1) When the detection object is caused to coexist with a temperature-responsive substance (i.e., a meltable substance), the temperature of the wells is raised to a temperature equal to or higher than the melting temperature of the meltable substance, or lowered to a temperature equal to or lower than the melting temperature thereof [when the below-described poly(N-isopropylacrylamide), which is solidified in response to a rise in temperature and is liquefied in response to a drop in temperature, is employed as a temperature-responsive substance, preferably, the temperature of a solution dispensed is lowered to the liquefaction temperature of poly(N-isopropylacrylamide) during the first dispensing; after the first dispensing, the detection object is brought into contact with a reaction solution dispensed through the second dispensing while the temperature of the wells is raised to the solidification temperature of poly(N-isopropylacrylamide); and the temperature of the wells is lowered to the liquefaction temperature of poly(N-isopropylacrylamide) during detection]. (2) When the detection object is caused to coexist with a gradually soluble substance, these substances are allowed to stand at room temperature in the wells of the detection instrument. Since a reaction solution is applied onto the surface of the detection instrument on which the wells are provided, even when a sealing material is provided as described above, air bubbles encounter difficulty in entering the sealed wells. In addition, there can be avoided the risk of erroneous data or a drop in detection sensitivity, which would otherwise occur as a result of contact between different detection objects via the reaction solution. Therefore, a liquid phase reaction can be performed on, for example, a microarray plate through a simple operation and with high accuracy.

The present invention also provides a detection kit employed for carrying out the present detection method (hereinafter the kit may be referred to as “the present detection kit”).

### BRIEF DESCRIPTION OF THE DRAWINGS

FIG. 1 is a schematic representation showing a detection plate.

FIG. 2 schematically shows the Invader assay.

FIG. 3 is a schematic representation showing a detection plate employed in an Example.

FIG. 4 is a photograph of a plate surface showing the results of a preliminary test of the present detection method.

FIG. 5 is a photograph of a plate surface showing the utility of the present detection method.

### BEST MODE FOR CARRYING OUT THE INVENTION

A. The Present Detection Method

As described above, in the present detection method, which employs a detection instrument having a plurality of wells at a surface thereof, a detection object is deposited onto the inner walls of the wells of the detection instrument; a reaction solution containing a substance reactive with the detection object is brought into contact with the inner walls of the wells of the detection instrument on which the detection object has been deposited; and a signal generated through this contact is detected for evaluation of the detection object.

(1) Detection Instrument

As used herein, “a detection instrument having a plurality of wells at a surface thereof” refers to a detection instrument having, at a surface thereof, wells in which a liquid phase reaction can be performed. Such a detection instrument generally refers to a microarray plate. In the present invention, a detection instrument employed generally has a plate-like form, but the form of the instrument is not necessarily limited thereto.

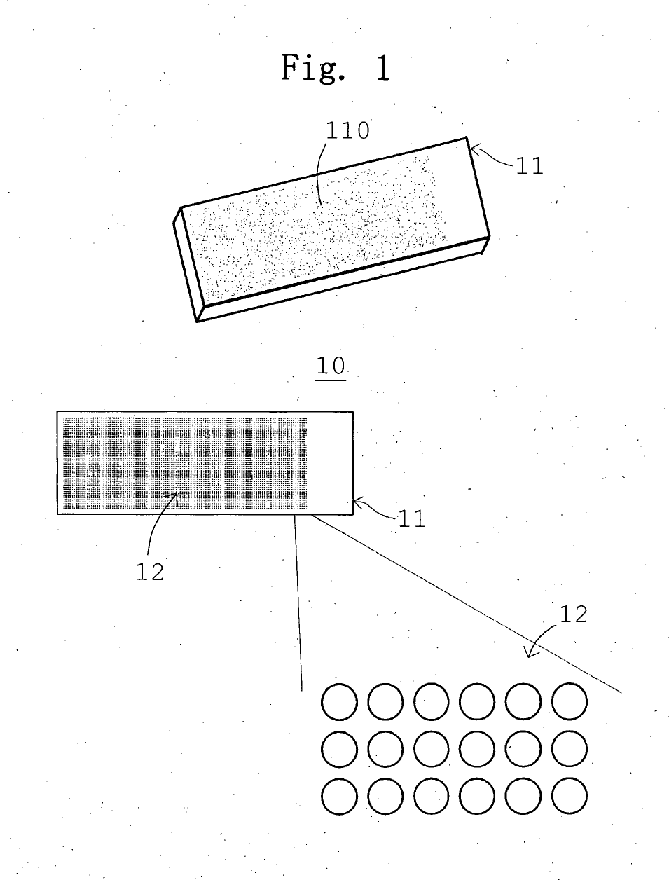

FIG. 1 shows an embodiment of a detection plate which is preferably employed in the present detection method. The detection plate 10 is produced by providing numerous (at least two) wells 12 at the surface 110 (only one of the two surfaces) of a base plate 11.

No particular limitation is imposed on the capacity of each of the wells 12, and the well capacity is appropriately determined in consideration of the volume of a liquid phase required for detection of a liquid phase reaction performed in the well. The capacity of each of the wells 12 must be appropriately greater than the volume of a liquid phase required for detection of a liquid phase reaction. Specifically, the well capacity is preferably about 100 to about 6,800% of the required liquid-phase volume.

In the detection plate 10, detection of a liquid phase reaction—the required volume of a liquid phase is preferably on the order of less than μL—is performed for each of the wells 12. Therefore, the capacity of each of the wells 12 is preferably 1 μL or less, more preferably about 0.01 μL or less. The preferred minimum capacity of each of the wells 12 should be determined in consideration of the detection sensitivity of a liquid phase reaction, as well as the technique for providing the wells 12.

In the detection plate 10, no particular limitation is imposed on the density of the wells 12 present in a unit area of the plate, and the well density is determined in consideration of the size of each of the wells 12, the technique for providing the wells 12, and the technique for detecting a liquid phase reaction. In general, the well density is preferably about 1 to about 40,000 wells/cm². In the case where the reaction performed in the detection plate is based on the below-described Invader assay, the well density is particularly preferably about 1 to about 10,000 wells/cm². In the case where the Invader assay is performed in a “light treatment” mode [i.e., in the case where the amount of DNAs to be detected is small or the number of single nucleotide polymorphisms (SNPs) to be detected is small; for example, in the case where several hundreds of subjects are subjected to testing and several SNPs are detected], the well density is more preferably one well/cm² or more and less than 400 wells/cm². Meanwhile, in the case where the Invader assay is performed in a “heavy treatment” mode [i.e., in the case where the number of single nucleotide polymorphisms (SNPs) to be detected is large (e.g., several tens of thousands of SNPs or more)], the well density is more preferably about 400 to about 10,000 wells/cm². In the case where the reaction performed on the detection plate is based on a low-density microarray assay (e.g., in the case where expression of several hundreds of genes is to be detected), or a liquid phase reaction other than the low-density Invader assay, such as immune response, radioimmunoassay, or homogeneous assay (e.g., in the case where several species are to be detected in the liquid phase reaction), the well density is particularly preferably one well/cm² or more and less than 400 wells/cm². In the case where the reaction performed on the detection plate is based on a high-density microarray assay (e.g., in the case where expression of several thousands to several tens of thousands of genes is to be detected), or a liquid phase reaction other than the high-density Invader assay (e.g., in the case where several thousands to several tens of thousands of species are to be detected in the liquid phase reaction), the well density is particularly preferably 400 to 40,000 wells/cm², more preferably 400 to 10,000 wells/cm².

No particular limitation is imposed on the shape of the wells 12, and the wells assume, for example, a semispherical shape, a semispherical-bottomed cylindrical shape, a cylindrical shape, a mortar-like shape, a conical shape, a pyramidal shape, or a rectangular columnar shape. The opening of each of the wells 12 is preferably has a dimension so that a liquid phase can be readily injected into the well. Specifically, the size of the opening is preferably about 0.01 to about 0.5 mm (in diameter).

No particular limitation is imposed on the material of the detection plate 10, so long as the material exhibits sufficient rigidity for practical use. Particularly when the liquid phase reaction detection means is detection means employing fluorescence, preferably, the detection plate is formed of a material exhibiting no autofluorescence, from the viewpoint of prevention of occurrence of background upon detection. In such a case, the detection plate 10 is formed of, for example, glass, ceramic, metallic, or plastic material.

Examples of thermoplastic resins (plastic materials) include a polymer having a main chain formed almost solely of carbon atoms. Specific examples of such a polymer include olefin polymers such as propylene polymers (e.g., polypropylene) and 4-methylpentene-1 polymers; cycloolefin polymers such as norbornene polymers (e.g., ethylene-norbornene copolymers); acrylic polymers such as methyl methacrylate polymers, copolymers of isobornyl methacrylate, and dicyclopentanylmethacrylic copolymers; styrene polymers such as amorphous styrene polymers, syndiotactic styrene polymers, para-t-butylstyrene polymers, α-methylstyrene-methyl methacrylate copolymers, and ABS resin; cyclohexyl malate polymers; dimethyl itaconate polymers; hardened vinyl chloride resin; fluorocarbon polymers (e.g., vinylidene fluoride polymers and tetrafluoroethylene polymers); and other vinyl polymers.

Examples of thermoplastic resins include a polymer having a main chain containing a hetero atom. Specific examples of such a polymer include polyacetal resin, polycarbonate, polysulfone, aromatic polyester, polyamide, polyurethane, polyphenylene ether, polyphenylene sulfide, polyimide resin, and triacetyl cellulose.

Examples of thermosetting resins include unsaturated polyester, epoxy resin (particularly, alicyclic epoxy resin), three-dimensional hardened polyurethane, unsaturated acrylic resin (including epoxy acrylate resin), melamine resin, three-dimensional styrene resin, three-dimensional silicone resin, and allyl resin (e.g., diallyl phthalate resin or diethylene glycol diallyl carbonate resin).

If necessary, the detection plate 10 formed of glass or plastic material may be subjected to surface treatment such as silicone treatment or fatty acid treatment through a customary method. Particularly, in many cases, the detection plate 10 is preferably subjected to silicone treatment, in order to prevent adsorption, onto the surface of the plate, of a detection material, a reagent, or the like.

Silicone treatment can be carried out through a customary method. For example, silicone treatment can be performed through the following procedure: a silicone raw material such as colloidal silica is hydrolyzed through application of the sol-gel process or a similar technique; a curing catalyst, a solvent, a leveling agent, and, if necessary, a UV absorbing agent or the like are added to the above-hydrolyzed product, to thereby prepare a silicone coating material; and the detection plate is coated with the resultant coating material through a customary technique; for example, preferably, dipping, vapor deposition, spraying, roll coating, flow coating, or spin coating.

The detection plate 10 may be colored, to thereby prevent, for example, autofluorescence of the plate in the case of detection employing fluorescence, and adverse effects caused by fluorescence emitted from adjacent wells. When coloring is performed, chromaticity, hue, brightness, etc. may be appropriately determined as desired. In general, the color is preferably black. When a black-color detection plate is produced, a black pigment such as carbon is mixed with the material of the plate.

No particular limitation is imposed on the specific size and shape of the detection plate 10, and the size and shape may be determined arbitrarily. However, preferably, the size and shape of the detection plate are determined on the basis of generally employed standards for the size and shape of microarray, from the viewpoint of practical use of the detection plate. Various microarray analysis apparatuses, analysis software, microarray-related dispensing apparatuses, etc. are designed in accordance with such standards for microarray, and therefore, the detection plate 10 is preferably designed to have a size and shape which meet with such standards. Specifically, the detection plate is preferably in the form of a plate having a size nearly equal to that of a glass slide which is generally employed in Japan (i.e., 26 mm in width×76 mm in length×1 mm in thickness). Similarly and also preferably, the shape and size of the detection plate 10 may be determined so as to be in agreement with the standards for the shape and size of microarray in regions in which the detection plate 10 will be employed [e.g., US (size: about 1 inch in width×about 3 inches in length) and Europe]. Alternatively, the detection plate preferably has a size suited to a dispensing or detection apparatus employed. The detection plate may have a size equal to that of microtiter plate, since a microarray scanner which can analyze a microarray chip having a microtiter plate size has been commercially available.

No particular limitation is imposed on the process for producing the detection plate 10, but the plate is generally produced through a process in which wells are provided directly onto a single plate.

In this production process, for example, a thin film which is formed of, for example, vinyl chloride and has numerous through holes corresponding to the wells 12, the thin film serving as a mask, is attached onto the surface of a non-treated plate, and microwells are formed on the mask-coated plate surface through sand blasting (a technique for forming wells on the plate surface by hitting microparticles onto the surface at high speed), die-cutting or embossing by means of a die having microirregularities, machining by means of a microdrill, or a similar technique. Through this production process, the detection plate 10 having, at a surface thereof, the numerous wells 12 can be produced.

When the detection plate 10 is formed of plastic material, embossing or die-cutting is preferably employed for forming wells.

The detection plate 10 can be produced through such a production process.

(2) Detection Object

No particular limitation is imposed on the detection object, so long as it can be detected through a liquid phase reaction. Specific examples of the detection object include, but are not particularly limited to, nucleic acids to be genetically analyzed (DNA and/or RNA, which may have a double- or single-stranded structure, or a specific three-dimensional structure (e.g., a hairpin structure)), polypeptides, antibodies, bacteria, viruses, and various clinical samples (e.g., blood samples, urine samples, lymph samples, synovial samples, and saliva samples).

Preferably, the detection object is caused to be contained in a solution for first dispensing (hereinafter the solution may be referred to as a “first dispensing solution”).

If necessary, the first dispensing solution may contain, in addition to a solvent (e.g., water, 2-propanol (which is a suitable solvent for the below-described DPPC), or isopropyl alcohol), an additive selected in consideration of the type of the detection object (e.g., a stabilizer, a treatment agent, or an immobilizing agent).

No particular limitation is imposed on the amount of the detection object contained in the first dispensing solution, and the detection object content should be determined in consideration of, for example, the type of the detection object or detection purposes. However, the detection object content must exceed a level such that a detectable signal can be generated through contact between the detection object and a substance reactive therewith, and must fall below a level such that noise is observed.

(3) Reaction Solution, etc.

A reaction solution can be appropriately selected in consideration of the type of the detection object employed, as well as the liquid phase reaction selected.

No particular limitation is imposed on the liquid phase reaction performed in the present invention. Examples of the liquid phase reaction include protein catalytic reaction such as enzyme reaction, antigen-antibody reaction, interaction between proteins, and specific affinity reaction between substances (including hybridization between nucleotide fragments).

Detection of the liquid phase reaction can be performed through means which is currently employed in microarray techniques. Specifically, for example, the detection plate in which the liquid phase reaction has been performed in the present detection method is subjected to analysis by means of a highly sensitive fluorescence scanner, whereby fluorescence in each of the wells of the detection plate can be detected. In addition, there can be performed, for example, detection by means of a radioisotope, detection through the EIA method, and homogeneous detection by means of AlphaScreen™ [product of PerkinElmer, Inc. (US)].

As described above, a most preferred mode of the liquid phase reaction performed in the present detection method is a reaction based on the Invader assay [Third Wave Technologies, Inc. (US)].

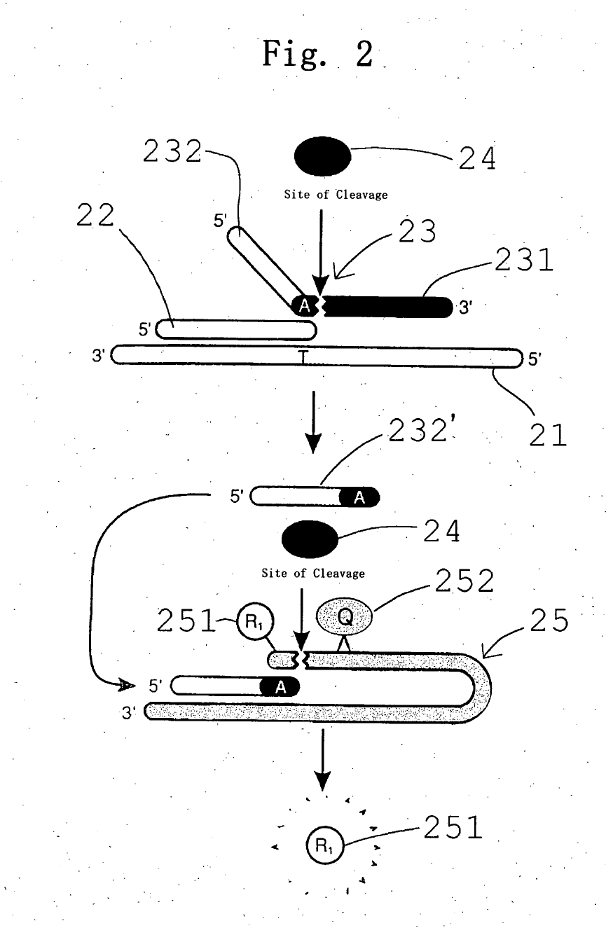

FIG. 2 schematically shows the basic feature of the Invader assay.

As shown in FIG. 2, firstly, a first nucleotide fragment 22 is hybridized with a nucleotide fragment (wild-type gene) 21 serving as a template.

The first nucleotide fragment 22—in which the base [A (adenine) in FIG. 2] complementary to the base to be detected for determination of mutation (hereinafter the base may be referred to as a “base for mutation detection”) [T (thymine) in FIG. 2 (i.e., wild type)] of the template nucleotide fragment 21 is located at the 3′-end—is complementary to the template nucleotide fragment 21. (In this case, the base at the 3′-end of the first nucleotide fragment 22 is not complementary to the base for mutation detection, but, when the 3′-end base interferes in association reaction between the base for mutation detection and a second nucleotide fragment, a locally three-base-overlapped structure is formed.)

Subsequently, a second nucleotide fragment 23 is hybridized with the locally double-stranded structure formed of the template nucleotide fragment 21 and the first nucleotide fragment 22.

The second nucleotide fragment 23 is a composite nucleotide fragment including a “complementary portion” 231 which is complementary to the template nucleotide fragment 21, and a “detection portion” 232 which has a detection element and is not complementary to the template nucleotide fragment, wherein the portion 231 is located on the 3′-side, and the portion 232 is located on the 5′-side so as to be continuous with the portion 231. The base located at the 5′-side end of the “complementary portion” 231 is (A) (i.e., a base complementary to the base for mutation detection (T)).

This second hybridization forms a locally three-base-overlapped structure including the base for mutation detection (T) of the template nucleotide fragment 21, the 3′-end base of the first nucleotide fragment 22, and the base (A) located at the 5′-side end of the “complementary portion” 231 of the second nucleotide fragment.

Subsequently, a nuclease 24 having activity to specifically cleave the locally three-base-overlapped structure on its 3′-side is caused to act on the structure, and a detection portion 232′ of the second nucleotide fragment 23 which has been cleaved by the nuclease [the 3′-end base of the portion 232′ is base (A), which is complementary to the base for mutation detection (T)] is detected, whereby the template nucleotide fragment 21 can be detected to be a wild type.

As shown in FIG. 2, when a hairpin-shaped probe (nucleotide fragment) 25 labeled with a fluorescent dye 251 in the vicinity of its 5′-end and with a quencher 252 in the vicinity of its 3′-side is caused to coexist with the aforementioned hybridization system, the aforementioned wild type can be detected.

A single-stranded portion (on the 3′-side) of the hairpin-shaped probe 25 is designed so as to be complementary to the detection portion 232 of the second nucleotide fragment 23. The base for mutation detection (T) is one base on the 5′-side which is adjacent to the base located at the 5′-side end of the single-stranded portion. When the detection portion 232′ is hybridized with the single-stranded portion of the hairpin-shaped probe 25, at the tip of a double-stranded portion of the probe 25, a locally three-base-overlapped structure is formed of the base (A) located at the 3′-end of the detection portion 232′ and the hairpin-shaped probe 25. The nuclease 24 acts on the locally three-base-overlapped structure, and the hairpin-shaped probe 25 is cleaved at a site between a portion labeled with the fluorescent dye 251 and a portion labeled with the quencher 252, whereby the portion labeled with the fluorescent dye 251 is released. Since the thus-released portion is no longer affected by the quencher 252, fluorescence emitted from the released portion can be detected. Through detection of the fluorescence, the template nucleotide fragment 21 can be detected to be a wild-type gene in which no mutation is observed at the base for mutation detection.

Meanwhile, in the case where the base for mutation detection of the template nucleotide fragment 21 is not a wild-type base (T) but an SNP base (e.g., G (guanine)), and the base G is positively detected, the complementary base of the first nucleotide fragment 22 and the second nucleotide fragment 23 is changed from the above-employed A to C (cytosine), which is complementary to G, and another sequence set including the sequence of the detection portion 232 and the corresponding sequence of the probe 25 is provided. In addition, the fluorescent dye 251 and the quencher 252 provided on the hairpin-shaped probe 25 are changed to a fluorescent dye which emits fluorescence differing from the above fluorescence and a quencher corresponding to the fluorescent dye, respectively, whereby the SNP of the template nucleotide fragment 21 can be detected by means of fluorescence emitted from the different fluorescent dye.

When the template nucleotide fragment 21 is a nucleotide fragment including a wild-type base and a mutated base; i.e., a hetero-type nucleotide fragment, the nucleotide fragment can be positively detected by means of a mixture of the aforementioned two types of fluorescence.

In the above-described embodiment, the detection system employs the hairpin-shaped probe. However, for example, the detection portion 232 can be directly labeled with a fluorescent dye or an isotope, and the thus-labeled detection portion can be directly detected, whereby SNPs, etc. can be detected. In the above-described embodiment, the base of the template nucleotide fragment is positively detected in both the case where the nucleotide fragment has SNPs and the case where the nucleotide fragment does not have SNPS. However, in either of the above cases, negative detection, in which a label such as fluorescence is not detected, can be performed.

In the above-described Invader assay, as reaction proceeds, the nuclease which specifically cleaves the locally three-base-overlapped structure continuously acts in a step where the “detection portion” of the second nucleotide fragment is cleaved, and in a step where a portion labeled with a fluorophore is separated from a portion labeled with a quencher (in the case where the hairpin-shaped probe is employed). Therefore, a label employed in the Invader assay, such as fluorescence, is sensitized; i.e., the Invader assay involves a very sensitive liquid phase reaction. When a micro liquid phase reaction like the case of the present invention is detected, employment of the Invader assay is most preferred. The Invader assay is very useful for efficiently detecting SNPS, which are a key to personalized medicine. When the present invention is applied to the Invader assay, SNPs can be easily detected in an efficient and exhaustive manner. Industrial significance of such detection of SNPs is very high.

In the case where the present detection method employs the Invader assay as detection means, the aforementioned template nucleotide fragment 21 is selected as a detection object which is caused to be contained in a first dispensing solution, and a solution containing the aforementioned detection element of the Invader assay is selected as a reaction solution.

As described above, in the present detection method, a first dispensing solution containing a detection object is dispensed into a plurality of wells provided on a detection instrument, followed by drying, to thereby deposit the detection object onto the inner walls of the wells; a reaction solution containing a substance reactive with the detection object is brought into contact with the inner walls of the wells of the detection instrument on which the detection object has been deposited; and a signal generated through this contact is detected, whereby the detection object can be evaluated. However, in this method, when second dispensing, sealing, and detection are rapidly performed with low accuracy, the contents of the wells could leak onto the surface of the detection instrument, and then mix with one another, which may adversely affect evaluation of the detection object.

Therefore, in the present invention, the detection object is caused to coexist with a specific substance; i.e., a temperature-responsive substance and/or a gradually soluble substance, on the inner walls of the wells. Thus, those skilled in the art can secure a period of time sufficient for the detection object to be retained in the wells after the second dispensing.

(4) Temperature-Responsive Substance and Gradually Soluble Substance

No particular limitation is imposed on the temperature-responsive substance employed, but the temperature-responsive substance is preferably a substance which, in the absence of an additive (e.g., a salt), undergoes change in phase from solid (gel) to liquid at a temperature of about 10 to about 90° C. (preferably about 40 to about 70° C.). Specific examples of such a substance include gelatin, agar, DPPC (dipalmitoylphosphatidylcholine), poly(N-isopropylacrylamide), and poly(ε-caprolactone). Gelatin is particularly preferred.

Examples of the gradually soluble substance employed include polyhydric alcohols, such as polysaccharides (e.g., dextran), starch syrup components (e.g., maltose and trehalose), polyethylene glycol, and xylitol. Trehalose is particularly preferred.

Generally, one or more temperature-responsive substances and/or one or more gradually soluble substances (hereinafter these two types of substances may be collectively called a “holding substance”) may be added to the first dispensing solution.

The amount of a holding substance contained in the first dispensing solution must be equal to or greater than a level such that the holding substance can sufficiently hold the detection object while being maintained in a dry state, a moisturized state, or a swollen state. When the amount of the holding substance—which is moisturized or swollen after the reaction solution is dispensed (second dispensing) after drying—is insufficient, the detection object is incompletely held in the holding substance, and thus released. When the wells of the detection instrument are in communication with one another via the reaction solution, the thus-released detection object could immediately enter adjacent wells, which is not preferred. Even when the wells of the detection instrument are filled with the reaction solution, if the holding substance is present in such an amount that it can sufficiently hold the detection object, the detection object is prevented from entering adjacent wells. Therefore, in a well which is separated from adjacent wells through sealing after dispensing of the reaction solution, a well-specific reaction (i.e., a reaction independent of the reaction in any of the adjacent wells) occurs. That is, release of the detection object due to change in phase of the temperature-responsive substance by heating or cooling after sealing, or due to gradual dissolution of the gradually soluble substance at room temperature is not affected.

When, for example, the temperature-responsive substance is gelatin, the amount of gelatin contained in the first dispensing solution is preferably 0.05 to 2 mass % on the basis of the entirety of the solution. When the amount of gelatin is less than 0.05% on the basis of the entirety of the solution, the detection object contained in a well may failed to be completely held in gelatin (i.e., a temperature-responsive substance) as described above, and the detection object eluted into the reaction solution would come into contact, via the reaction solution, with the detection object contained in another well, resulting in a drop in detection sensitivity. In contrast, when the amount of gelatin exceeds 2 mass %, the first dispensing solution itself becomes excessively viscous, which tends to cause a problem in terms of dispensing.

Preferably, the first dispensing solution is prepared by dissolving a detection object in an aqueous solution of a holding substance, which is generally in the form of liquid (sol). However, the first dispensing solution is not necessarily prepared through this method, but may be prepared through another method. The first dispensing solution may be prepared immediately before being dispensed into wells of a detection plate, or may be prepared several days to several hours before being dispensed. The period of time for storing the first dispensing solution is preferably determined in consideration of the stability, etc. of the detection object. The first dispensing solution must be in the form of liquid at least at the time when it is dispensed. It is not desirable that, after the detection object is added to the first dispensing solution, the solution is maintained at a temperature at which the detection object is denatured.

(5) Detection Method

The present detection method is performed as follows. A first dispensing solution in the form of liquid is dispensed into wells of the aforementioned detection instrument (plate) (dispensing of the solution may be performed manually, but is preferably performed by means of a microdispenser (e.g., an inkjet-type microdispenser)), and the first dispensing solution contained in the wells is subjected to, for example, a drying treatment (which is preferably performed when a gradually soluble substance is employed as a holding substance, or when no holding substance is employed) or a cooling treatment (including a treatment in which the solution is allowed to stand at room temperature, which treatment is preferably performed when a temperature-responsive substance is employed as a holding substance), to thereby deposit the solution onto the wells of the detection instrument. Thereafter, a reaction solution containing a substance reactive with a detection object contained in the first dispensing solution is brought into contact with the inner walls of the wells on which the first dispensing solution has been deposited, and the detection object is released through dissolution thereof in the reaction solution by, for example, raising or lowering the temperature of the wells, or allowing the detection instrument to stand [when the above-described poly(N-isopropylacrylamide), which is solidified in response to a rise in temperature and is liquefied in response to a drop in temperature, is employed as a temperature-responsive substance, preferably, the temperature of the first dispensing solution is lowered to the liquefaction temperature of poly(N-isopropylacrylamide) during dispensing of the first dispensing solution; after dispensing of the first dispensing solution, the detection object is brought into contact with the reaction solution while the temperature of the wells is raised to the solidification temperature of poly(N-isopropylacrylamide); and the temperature of the wells is lowered to the liquefaction temperature of poly(N-isopropylacrylamide) during detection of the detection object]. Detection of the thus-generated signal enables the detection object to be evaluated.

In a mode wherein the reaction solution is brought into contact with the first dispensing solution, efficiently and preferably, the reaction solution is uniformly applied onto the plate surface after the first dispensing solution has been deposited onto the inner walls of the aforementioned wells.

B. The Present Detection Kit

The present detection kit, which is employed for carrying out the present detection method, comprises the following elements a, b, and c:

a. a temperature-responsive substance and/or a gradually soluble substance;

b. a reaction solution or a solute of the solution; and

c. a detection instrument (plate) having a plurality of wells.

The present detection kit comprising the aforementioned elements is employed, for example, as follows: a collected detection object is added to a mixture of a solvent (e.g., water) and a temperature-responsive substance and/or a gradually soluble substance (i.e., a holding substance(s)), to thereby prepare a first dispensing solution; the first dispensing solution is dispensed into wells of a detection plate included in the kit, followed by solidification of the solution; and a detection reaction is performed through the aforementioned procedure of the present detection method by use of a reaction solution included in the kit, whereby the detection object can be evaluated.

If necessary, the present detection kit may contain an additional element; for example, a solvent employed for dissolving the aforementioned holding substance (e.g., purified water or 2-propanol), a preservative for a detection object, or a stabilizer.

When the present detection kit is employed in the Invader assay, the detection object is a nucleic acid, and the reaction solution is a solution which enables the Invader assay reaction to be performed on the nucleic acid.

### EXAMPLES

The present invention will next be described in more detail by way of Examples, which should not be construed as limiting the invention thereto.

Detection Plate

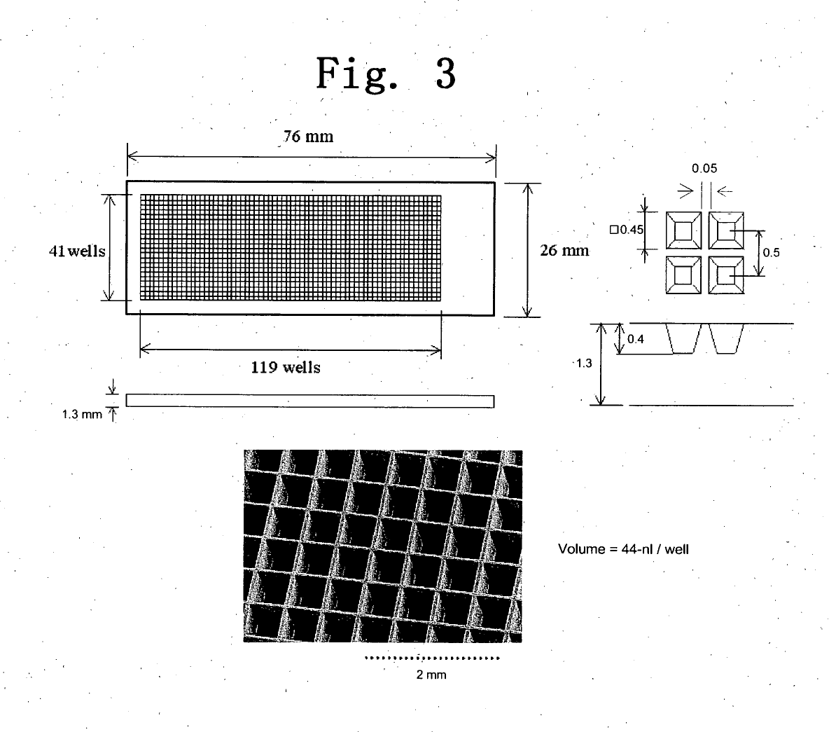

A detection plate employed in the present Example was produced through the above-described production process. FIG. 3 schematically shows the detection plate. The detection plate shown in FIG. 3 is formed of a polycarbonate slide (plastic slide) having wells at high density. The detection plate was formed by providing 5040 wells (each having an opening size of 0.45×0.45 (mm) and a depth of 0.45 (mm)) on the plastic slide having a size of 22×76×1 (mm). A region of the detection plate on which the wells are provided has an area of 21×60 (mm), and each of the wells has a capacity of 44 nL. Adjacent wells are separated by a partition wall whose upper portion has a thickness of 0.05 mm.

A detection object was dispensed into each of the wells by means of an inkjet-type microdispenser (synQUAD™, product of Cartesian).

Determination of Appropriate Gelatin Concentration

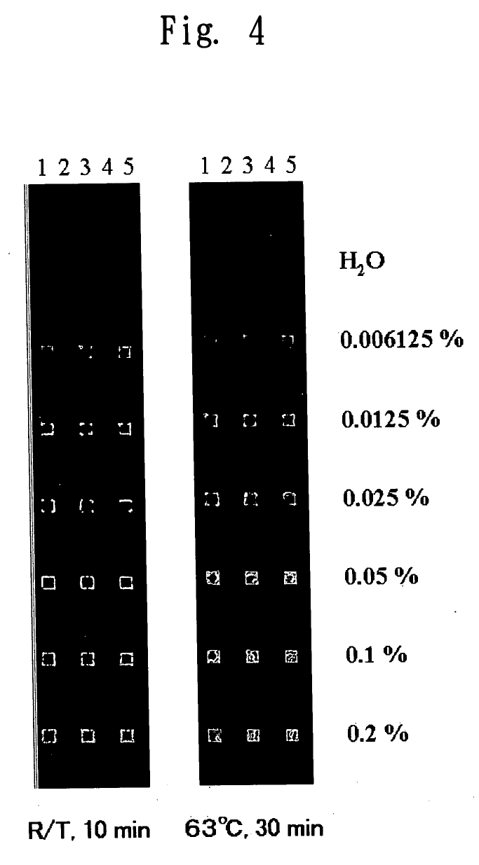

Human genomic DNA and 0.1% gelatin were dispensed into each of the wells of the detection plate shown in FIG. 3 at room temperature, followed by solidification of the gelatin. Thereafter, a 10,000-fold-diluted OliGreen 1% TritonX-100 solution, serving as a reaction solution, was applied in one time onto the plate surface, to thereby dispense the reaction solution into the wells, followed by DNA staining reaction. FIG. 4 shows the state of the plate surface after reaction. In FIG. 4, “%” corresponds to gelatin concentration. In the wells in which the DNA was dispensed (i.e., the wells present at points where rows 1, 3, and 5 are crossed with columns marked with H₂O and gelatin concentrations), strong fluorescence was emitted through Oligreen staining of the DNA. In addition, fluorescence was observed in some wells adjacent to the DNA-dispensed wells.

The shape formed by staining was found to vary with the concentration of gelatin mixed with the DNA. Specifically, in the wells in which the DNA was not eluted from the surfaces of the inner walls into the reaction solution (i.e., the wells containing gelatin at a concentration of 0.05% or more), the DNA deposited on the inner walls was stained to form an angular-doughnut-like shape. In contrast, in the wells containing gelatin at a concentration of 0.025% or less, the DNA was eluted from the inner walls into the reaction solution, and a fluorescence signal was observed at interior portions of the wells other than the inner walls. In addition, a fluorescence signal was observed at wells adjacent to the DNA-dispensed wells. ScanArray 5000 (product of PerkinElmer, Inc.) was employed as a fluorescence scanner (focal depth: 0.1 mm as measured from the plate surface). The human genomic DNA was heat-denatured before use.

As is clear from the thus-obtained data, a reaction solution can be dispensed into a well containing genomic DNA mixed with gelatin (concentration: 0.05% or more) without causing any effect on a well adjacent to the DNA-containing well.

The Present Detection Method

The present detection method was carried out on the basis of the results shown in FIG. 4. Specifically, three 50-ng/μL genomic DNA samples having different genotypes, which were caused to coexist with 0.1% gelatin, were dispensed into different wells (20 nL for each well), followed by solidification of the gelatin at room temperature. Thereafter, through the procedure of the method shown in FIG. 3, an invader reagent [i.e., a mixture of an enzyme, probe mix (for determination of ABCA1 gene-447C/T polymorph), an FRET reagent, and a magnesium solution] was collectively applied onto the plate for dispensing of the reagent. After completion of reaction, analysis was performed by means of the aforementioned fluorescence scanner.

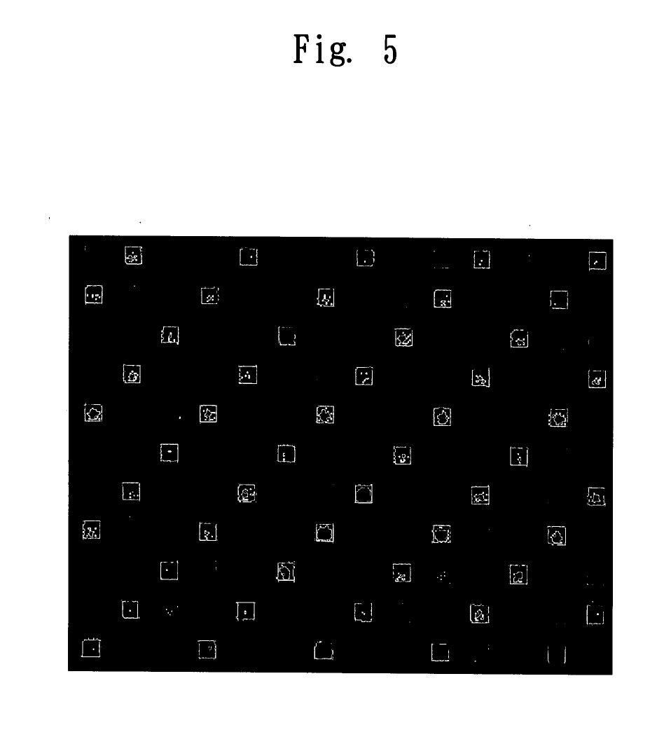

FIG. 5 shows the state of the plate surface after reaction. In FIG. 5, green wells correspond to wells in which T-homozygotic genomic DNA was dispensed; red wells correspond to wells in which C-homozygotic genomic DNA was dispensed; and yellow wells correspond to wells in which heterozygotic genomic DNA was dispensed (in place of FIG. 5 (i.e., monochromatic image), a color reference image has been prepared for submission). There was not observed erroneous determination due to, for example, mixing of the reagents contained in different wells.

Thus, the present detection method (i.e., detection by means of the aforementioned detection plate) was found to be a rapid, simple, and inexpensive method; i.e., the method does not require a special apparatus; the method does not require careful adjustment of the positions of the tips of outlets of a microdispenser in accordance with the positions of wells of the plate during the course of dispensing; and the method employs a system in which each of the wells is filled with a dispensing solution, and thus generates a few errors.

### INDUSTRIAL APPLICABILITY

According to the present invention, there is provided means for efficiently performing a liquid phase reaction on a detection plate (e.g., a microarray plate) without causing any problem in terms of generation of air bubbles in microwells.

1. A detection method employing a detection instrument having a plurality of wells at a surface thereof, the method comprising depositing a detection object onto the inner walls of the wells of the detection instrument; bringing a reaction solution into contact with the inner walls of the wells of the detection instrument on which the detection object has been deposited, the reaction solution containing a substance reactive with the detection object; and detecting a signal generated through this contact, to thereby evaluate the detection object.

2. A detection method according to claim 1, wherein the detection object deposited on the inner walls of a plurality of the wells at the surface of the detection instrument is caused to coexist with (1) a substance which, when being itself or in the form of a mixture with a solvent, undergoes change in phase from solid to liquid at about 10 to about 90° C., and/or (2) a substance which is gradually dissolved in a solvent at room temperature; and the substance (1) and/or the substance (2) is dissolved in the reaction solution during detection.

3. A detection method according to claim 2, wherein the substance (1); i.e., a substance which, when being itself or in the form of a mixture with a solvent, undergoes change in phase from solid to liquid at about 10 to about 90° C., is one or more species selected from the group consisting of gelatin, agar, DPPC (dipalmitoylphosphatidylcholine), poly(N-isopropylacrylamide), and poly(ε-caprolactone)

4. A detection method according to claim 2, wherein the substance (1); i.e., a substance which, when being itself or in the form of a mixture with a solvent, undergoes change in phase from solid to liquid at about 10 to about 90° C., is gelatin.

5. A detection method according to claim 2, wherein the substance (2); i.e., a substance which is gradually dissolved in a solvent at room temperature, is one or more species selected from the group consisting of dextran, xylitol, polyethylene glycol, maltose, and trehalose.

6. A detection method according to claim 2, wherein the substance (2); i.e., a substance which is gradually dissolved in a solvent at room temperature, is trehalose.

7. A detection method according to claim 1, wherein the reaction solution is brought into contact with the inner walls of the wells provided at the surface of the detection instrument by applying the reaction solution onto the side surfaces of openings of the wells of the detection instrument.

8. A detection method according to claim 1, wherein the detection object is a nucleic acid.

9. A detection method according to claim 8, wherein the reaction solution is a reaction solution which enables the Invader assay reaction to be performed on the nucleic acid.

10. A detection method according to claim 1, wherein the wells of the detection instrument are provided at a density of 1 to 40,000 wells/cm².

11. A detection method according to claim 1, wherein the detection instrument is in the form of a plate.

12. A detection kit employed for carrying out a detection method as recited in claim 1, the kit comprising the following elements A, B, and C:
A. (1) a substance which, when being itself or in the form of a mixture with a solvent, undergoes change in phase from solid to liquid at about 10 to about 90° C., and/or (2) a substance which is gradually dissolved in a solvent at room temperature;
B. a reaction solution or a solute of the solution; and
C. a detection instrument having a plurality of wells.

13. A detection kit according to claim 12, wherein the detection object is a nucleic acid, and the reaction solution is a reaction solution which enables the Invader assay reaction to be performed on the nucleic acid.
